(19) United States
(12) Patent Application Publication
(10) Pub. No.: US 2021/0196530 A1
(43) Pub. Date: Jul. 1, 2021

(54) Absorbent Articles

(71) Applicant: 3M INNOVATIVE PROPERTIES COMPANY, St. Paul, MN (US)

(72) Inventors: Volker Hauschildt, Hilden (DE); Kristina Gaspers, Bedburg (DE)

(21) Appl. No.: 17/048,125

(22) PCT Filed: Apr. 26, 2019
(86) PCT No.: PCT/IB2019/053454

§ 371 Date: Oct. 16, 2020

(30) Foreign Application Priority Data
Apr. 26, 2018 (GB) 1806839.5

(51) Int. Cl. A61F 13/475; A61F 13/494

(52) U.S. Cl. CPC A61F 13/4757; A61F 13/4756; A61F 13/4758; A61F 13/49466; A61F 13/4946; A61F 13/49446; A61F 13/49426

(57) ABSTRACT

The present invention relates to an absorbent article comprising: topsheet; a backsheet; an absorbent core disposed in the central region of the absorbent between the top sheet and the backsheet; at least one elongate liquid response barrier member provided on an interior surface of the absorbent article in the peripheral region of the absorbent article and positioned along an outer edge of the absorbent article, either adjacent to or spaced apart from said outer edge, the at least one elongate liquid response barrier member comprising absorbent material and being configured and arranged such that the absorbent material expands upon absorbing liquid and upon expansion of the absorbent material the at least one elongate liquid response barrier member forms a barrier or a seal between the at least one elongate liquid response barrier member and a portion of the body of a wearer of the absorbent article; and wherein the absorbent article further comprises at least one elongate liquid conducting member extending from the central region of the absorbent article to the at least one elongate liquid response barrier member.

### TECHNICAL FIELD

The present invention relates to absorbent articles, e. g. disposable absorbent articles, such as diapers, incontinent briefs, training pants and the like.

### BACKGROUND

Infants and other incontinent individuals wear absorbent articles such as diapers to receive and contain urine and other body exudates. In general, absorbent articles function contain the discharged materials and/or to isolate these materials from the body of the wearer and from the wearer's garments and surroundings. Accordingly, it is desirable to completely eliminate or at least minimize leakage from the absorbent article.

In an effort to control leakage from absorbent articles and enhance containment of waste material, absorbent articles have been provided with elastic features, such as elastic waist and/or leg features. Despite such improvement such absorbent articles still show a tendency towards leakage. For example, it has been observed that absorbent articles having elastic waist features have a tendency to sag or gap away from the body of the wearer during wear or the waist feature has a tendency to roll down or in, all of which can lead to leakage about the wearer in the waist regions.

Containment flaps and moisture responsive members have also been proposed and/or utilized in absorbent articles. For example, U.S. Pat. No. 5,935,118 discloses an absorbent article includes a garment shell and at least one liquid containment beam formed of an absorbent material and bonded along an attachment edge to the garment shell so that the containment beam can lie against the garment shell and also pivot about an axis defined by the attachment edge, wherein the containment beam, for example, may swell upon absorbing liquid and thereafter form a physical barrier to inhibit liquid movement within the absorbent article. U.S. Pat. No. 7,314,967, for example, discloses a disposable absorbent article having first longitudinal edge, a second longitudinal edge, a front waist edge, and a rear waist edge, the disposable article comprising: a backsheet; a topsheet bonded to the backsheet; an absorbent core disposed between the topsheet and the backsheet; and a first moisture responsive member containing e. g. an absorbent gelling material and attached to the disposable absorbent article along at least one of the first longitudinal edge, the second longitudinal edge, the front waist edge, and the rear waist edge of the disposable absorbent article outboard of the absorbent core, wherein the first moisture responsive member provides contact with a portion of a wearer's body such that when the moisture responsive member is wetted by moisture from the wearer's body, the first moisture responsive member expands against the portion of the wearer's body to form a seal between the first moisture responsive member and the portion of the wearer's body.

### SUMMARY

Such absorbent articles still have an undesirable tendency towards leakage. In particular it has been found that for such absorbent articles including a containment flap or moisture responsive member outboard of the absorbent core, leakage often results from discharged liquid and materials, in particular in the case of discharge of large amounts of liquids and materials in a short period of time, running over the containment flap and/or the moisture responsive member before absorbent material of the flap or member has a chance to expand and thus before the flap or member has a chance to form a barrier or seal.

Therefore, there exists a need for an absorbent article to have improved leakage characteristics in particular an absorbent article that can provide maximum leakage protection when the leakage protection is needed the most, such as situations where there is excessive discharge of liquid and materials in a short period of time.

It is has been found that by providing one or more liquid conducting members extending from the central region of the absorbent article, which includes the absorbent core, to a liquid response barrier member comprising absorbent material, such as a containment flap or moisture responsive member, located outboard of the absorbent core in a peripheral region of the absorbent article, the absorbent material of the liquid response barrier member expands as a result of liquid absorbance and thus the liquid response barrier member forms a barrier or a seal before the overall level of discharged liquid and material within the absorbent article reaches the fluid response barrier member. In this manner, the formation of a barrier or seal before the amount of the discharged liquid and material in the absorbent article reaches a critical level is facilitated, and accordingly once the amount of the discharged liquid and material reaches a critical level the formed barrier or seal facilitates containment of discharged liquid and material within the absorbent article and thereby minimizing leakage.

Accordingly, the present invention provides an absorbent article having a central region and a peripheral region, the absorbent article comprising:
a liquid permeable topsheet that is located towards the wearer with the absorbent article is in use;
a backsheet;
an absorbent core disposed in the central region between the top sheet and the backsheet;
at least one elongate liquid response barrier member disposed on an interior surface of the absorbent article in the peripheral region and positioned along an outer edge of the absorbent article, either adjacent to or spaced apart from said outer edge, the at least one elongate liquid response barrier member comprising absorbent material and being configured and arranged such that the absorbent material expands upon absorbing liquid and upon expansion of the absorbent material the at least one elongate liquid response barrier member forms a barrier or a seal between the at least one elongate liquid response barrier member and a portion of the body of a wearer of the absorbent article; and
at least one elongate liquid conducting member extending from the central region of the absorbent article to the at least one elongate liquid response barrier member.

Favorably, the first end of the at least one elongate liquid conducting member positioned in the central region or directly adjacent to the outer periphery of the central region and the second end or a portion including the second end (“second end portion”) of the at least one elongate liquid conducting member is positioned adjacent or in proximity to the at least one elongate liquid response barrier member such that fluid that is conducted along the at least one elongate liquid conducting member is communicated to the absorbent material of the at least one elongate liquid response barrier member.

The at least one elongate liquid conducting member may be disposed either between the topsheet and the backsheet or on the interior surface of the absorbent article, for example onto the topsheet of the article, which allows for flexibility in the design of absorbent article as well as in the manufacture thereof. Alternatively, the liquid conducting member may be disposed between the topsheet and the absorbent core of the absorbent article.

It has been found advantageous to use liquid conducting members which comprise a fluid control film having at least one microstructure-bearing surface with a plurality of channels therein that permit transport of liquid between a central portion and the at least one elongate liquid response barrier member. Such elongate liquid conducting members may favorably comprise a cap layer to enclose the channels along at least a portion of the length of the liquid conducting member, for example to enhance the creation of discrete channels and/or to minimize or prevent liquid from being transferred over the sides of the liquid conducting members along its length onto the topsheet in the peripheral region.

Further scope of applicability and advantages of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limiting of the present invention, and wherein:

FIG. 1 represents a schematic, plan view of a first exemplary embodiment of an absorbent article according to the invention showing the body side of the absorbent article in a flat condition.

FIG. 2A represents a cross-sectional cutaway view generally from the plane of the 2-2 in FIG. 1 showing the liquid response barrier member and an end portion of a liquid conducting member.

FIG. 2B represents a cross-sectional cutaway view generally from the plane of the 2-2 in FIG. 1 showing a second variant of the liquid response barrier member and end portion of a liquid conducting member.

FIG. 2C represents a cross-sectional cutaway view generally from the plane of the 2-2 in FIG. 1 showing a third variant of the liquid response barrier member and end portion of a liquid conducting member.

FIG. 2D represents a cross-sectional cutaway view generally from the plane of the 2-2 in FIG. 1 showing a fourth variant of the liquid response barrier member and end portion of a liquid conducting member.

FIG. 3 represents a cross-sectional cutaway view generally from the plane of the 3-3 in FIG. 2A.

FIG. 4 represents a cross-sectional cutaway view generally from the plane of the 4-4 in FIGS. 2B and C.

FIG. 5 represents a cross-sectional cutaway view generally from the plane of the 5-5 in FIG. 2D.

FIGS. 6A through 6H are cross-sectional cutaway views of illustrative embodiments of fluid control films.

FIGS. 7A and 7B are schematic diagrams used to illustrate interaction of a liquid on a surface.

FIG. 8 represents a schematic, plan view of a second exemplary embodiment of an absorbent article according to the invention showing the body side of the absorbent article in a flat condition.

FIG. 9A represents a cross-sectional cutaway view generally from the plane of the 6-6 in FIG. 8 showing the liquid response barrier member and an end portion of a liquid conducting member.

FIG. 9B represents a cross-sectional cutaway view generally from the plane of the 6-6 in FIG. 8 showing a second variant the liquid response barrier member and an end portion of a liquid conducting member.

FIG. 10 represents a cross-sectional cutaway view generally from the plane of the 10-10 in FIGS. 9A and 9B.

Corresponding parts are marked with the same reference symbols in all figures.

### DETAILED DESCRIPTION

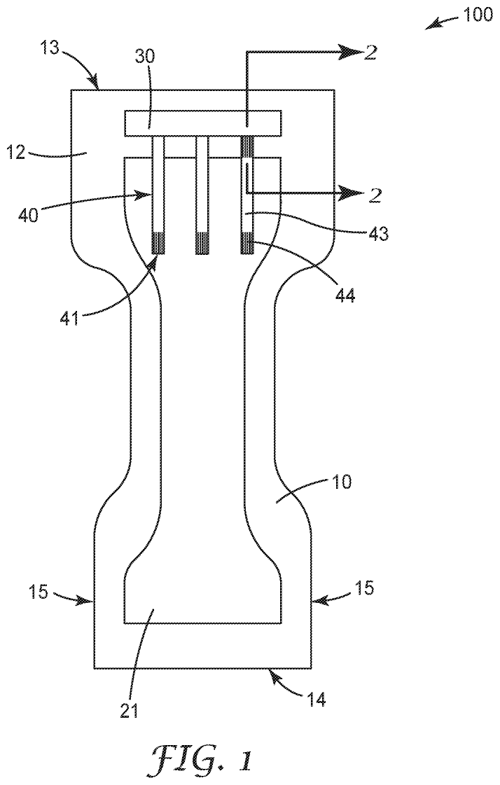

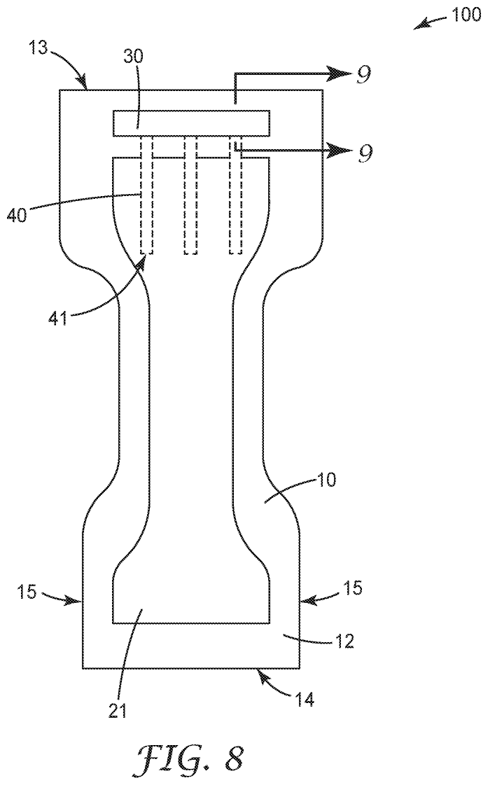

With reference to the FIGS. 1 and 8, absorbent articles formed according to the invention are shown for purposes of illustration as a disposable diaper. The invention may also be embodied into other types of absorbent articles such as other adult care products, e. g. undergarment for adult incontinence, training pants, feminine hygiene products or other personal care or health care garments.

Referring to FIGS. 1 and 8, in general the absorbent article 100 includes a topsheet 10, in particular a liquid permeable topsheet; a backsheet (11, not visible), in particular a liquid impermeable backsheet; a central region 21, wherein an absorbent core (20, not visible) is disposed at a region between the topsheet and the backsheet; a peripheral region 12 surrounding the central region and extending to outer edges of the absorbent article. FIG. 1 show a disposable diaper including for instance a rear waist edge 13, front waist edge 14, and two longitudinal edges 15. For the purposes of this invention, any suitable material known in the art may be used for the topsheet, backsheet and absorbent core. The term “disposed” is used to mean that an element(s) is formed (joined and positioned) in a particular place or position as a unitary structure with other elements or as a separate element joined to another element.

The absorbent article includes an elongate liquid response barrier member provided on an interior surface of the absorbent article (for example attached to the topsheet) in the peripheral region, and thus outboard of the absorbent core, and positioned along an outer edge of the absorbent article, either adjacent to or spaced apart from said outer edge. In the embodiments illustrated in FIGS. 1 and 8, the absorbent article 100 includes an elongate liquid response barrier member 30 disposed near the rear waist edge. In alternative embodiments, the elongate liquid response barrier member may be disposed near the front waist edge or near a longitudinal edge or alternatively the absorbent article may include two or more elongate members disposed e. g. near the front and waist edges or near all the edges. It will be appreciated that the absorbent articles according to the invention may further include other features, such as leg cuffs, elastic leg cuffs or elastic waist band. In such embodiments where for example the absorbent article includes elastic waist features and liquid response barrier member(s) disposed near to rear and/or front waist edge it is favorable to position the liquid response barrier member(s) inboard of the elastic waist feature.

The elongate liquid response barrier member comprises absorbent material. And in use the absorbent material expands upon absorbing liquid (e. g. as urine) and upon expansion of the absorbent material the elongate liquid response barrier member forms a barrier or a seal between the at least one elongate liquid response barrier member and a respective portion of the body of a wearer of the absorbent article.

In addition, the absorbent article includes at least one elongate liquid conducting member extending from the central region of the absorbent article to the at least one elongate liquid response barrier member. In the exemplary embodiments illustrated in FIGS. 1 and 8, the absorbent article 100 includes three elongate liquid conducting members 40 extending from the central region 21 of the absorbent article to the elongate liquid response barrier member 30. In the illustrated embodiment of FIG. 1 liquid conducting members 40 are provided on the interior surface of the absorbent article, e. g. attached to the topsheet. In alternative embodiments, the liquid conducting members may be disposed between the topsheet and backsheet, for example as indicated in the illustrated exemplary embodiment of FIG. 8.

One end of the elongate liquid conducting member may be positioned either directly adjacent to the outer periphery of the central region or in the central region. It has been found to be advantageous to the position the first end of the elongate liquid conducting member within the central region to facilitate the conduct of the fluid from the central portion to the liquid response barrier member in a timely manner. Favorably the positioning of the first end of the elongate liquid conducting member can be optimized having regard to the particular absorbent article under consideration, for example its construction, absorbency capabilities as well as what is considered to amount to a critical level of discharge fluid and material in terms of potential imminent leakage. For example, in a disposable diaper, discharged fluid and material reaching a level of about one-half up the rear portion of the diaper would typically be considered as critical and in such as case it would be desirable to dispose the first end of the elongate liquid conducting member within the central portion at position lower than said critical level. In the exemplary embodiments shown in FIGS. 1 and 8, it can be seen that the first ends 41 of the elongate liquid conducting members 40 are positioned in the central region on what will be the rear side of disposable diaper, in particular about level that corresponds to approximately two-thirds down the rear side of the diaper when the diaper is on the wearer. The second end of the at least one elongate liquid conducting member favorably is positioned adjacent or in proximity to the at least one elongate liquid response barrier member such that fluid that is conducted along the elongate liquid conducting member is communicated to the absorbent material of the at least one elongate liquid response barrier member. This will be discussed in more detailed below.

Elongate liquid conducting members favorably comprise a fluid control film having at least one microstructure-bearing surface with a plurality of channels therein that permit transport of liquid. Suitable fluid control films for use in the present invention are described in U.S. Pat. Nos. 5,514,120 and 5,728,446 and described generally in the following. The microreplicated channels of the fluid control films described in U.S. Pat. Nos. 5,514,120 and 5,728,446 are precisely replicated from a predetermined pattern and form a series of individual open capillary channels that extend along a major surface. (“Microreplication” means the production of a microstructured surface through a process where the structured surface features retain an individual feature fidelity during manufacture). These microreplicated channels formed in films are favorably uniform and regular along substantially each channel length and from channel to channel and advantageously provide more effective liquid flow than is achieved with webs, foam, or fibrous material, such as tows and nonwovens.

Desirably fluid control films used in the present invention are capable of spontaneously and uniformly transporting liquids along the axis of the film channels. Two general factors that influence the ability of fluid control films to spontaneously transport liquids (e. g., water, urine or vaginal secretions) are (i) the geometry or topography of the surface (capillarity, shape of the channels) and (ii) the nature of the film surface (e. g., surface energy). To achieve the desired amount of fluid transport capability the designer may adjust the structure or topography of the fluid control film and/or adjust the surface energy of the fluid control film surface. In order for a closed channel wick made from a fluid control film to function it preferably is sufficiently hydrophilic to allow the desired fluid to wet the surface. Generally, to facilitate spontaneous wicking in open channels, the fluid must wet the surface of the fluid control film, and the contact angle be equal to or less than 90 degrees minus one-half the notch angle.

The channels of fluid control films can be of any geometry that provides desired liquid transport, and preferably one which is readily replicated. The geometry of the channels may change over their length. Furthermore, the number of channels may vary, for example, the number may be 1 to 150, preferably 10 to 100, more preferred 20 to 80 channels.

Fluid control films can be formed from any thermoplastic materials suitable for casting, or embossing including, for example, polyolefins, polyesters, polyamides, poly (vinyl chloride), polyether esters, polyimides, polyesteramide, polyacrylates, polyvinylacetate, hydrolyzed derivatives of polyvinylacetate, etc. Polyolefins are preferred, particularly polyethylene or polypropylene, blends and/or copolymers thereof, and copolymers of propylene and/or ethylene with minor proportions of other monomers, such as vinyl acetate or acrylates such as methyl and butylacrylate. Polyolefins are preferred because of their excellent physical properties, ease of processing, and typically lower cost than other thermoplastic materials having similar characteristics. Polyolefins readily replicate the surface of a casting or embossing roll. They are tough, durable and hold their shape well, thus making such films easy to handle after the casting or embossing process. Hydrophilic polyurethanes are also preferred for their physical properties and inherently high surface energy. Alternatively, fluid control films can be cast from thermosets (curable resin materials) such as polyurethanes, acrylates, epoxies and silicones, and cured by exposure to heat or UV or E-beam radiation, or moisture. These materials may contain various additives including surface energy modifiers (such as surfactants and hydrophilic polymers), plasticizers, antioxidants, pigments, release agents, antistatic agents and the like.

The surface properties, e. g. the hydrophilicity, may vary over the length of the material such that the hydrophilicity increases or decreases from one end to another to form a gradient.

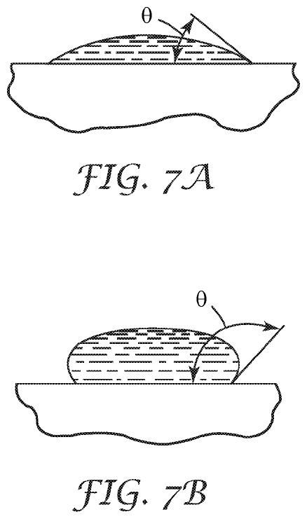

Generally, the susceptibility of a solid surface to be wet out by a liquid is characterized by the contact angle that the liquid makes with the solid surface after being deposited on the horizontally disposed surface and allowed to stabilize thereon. It is sometimes referred to as the “static equilibrium contact angle”, sometimes referred to herein merely as “contact angle”. As shown in FIGS. 7A and 7B, the contact angle Theta is the angle between a line tangent to the surface of a bead of liquid on a surface at its point of contact to the surface and the plane of the surface. A bead of liquid whose tangent was perpendicular to the plane of the surface would have a contact angle of 90. Typically, if the contact angle is 90 or less, the solid surface is considered to be wet by the liquid. Surfaces on which drops of water or aqueous solutions exhibit a contact angle of less than 90 are commonly referred to as “hydrophilic”. As used herein, “hydrophilic” is used only to refer to the surface characteristics of a material, i. e., that it is wet by aqueous solutions, and does not express whether or not the material absorbs aqueous solutions. Accordingly, a material may be referred to as hydrophilic whether or not a sheet of the material is impermeable or permeable to aqueous solutions. Thus, hydrophilic films used in fluid control films used in the present invention may be formed from films prepared from resin materials that are inherently hydrophilic, such as for example, poly (vinyl alcohol). Liquids which yield a contact angle of near zero on a surface are considered to completely wet out the surface. Polyolefins, in contrast, are typically inherently hydrophobic, and the contact angle of a polyolefin film, such as polyethylene or polypropylene, with water is typically greater than 90.

Depending on the nature of the microreplicated film material itself, and the nature of the fluid being transported, one may desire to adjust or modify the surface of the film in order to ensure sufficient capillary forces. For example, the surface of the fluid control film may be modified in order to ensure it is sufficiently hydrophilic. Here body liquids that will come into contact with the fluid control films are aqueous. Thus, if films made of a polyolefin, for example, are to be used as fluid control films, they generally must be modified, e. g., by surface treatment, application of surface coatings or agents, or incorporation of selected agents, such that the surface is rendered hydrophilic so as to exhibit a contact angle of 90 or less, thereby enhancing the wetting and liquid transport properties of the fluid control film. Suitable methods of making the surface hydrophilic include: (i) incorporation of a surfactant; (ii) incorporation or surface coating with a hydrophilic polymer; and (iii) treatment with a hydrophilic silane.

Other methods are also envisioned.

The fluid control films may have a variety of topographies. Preferred fluid control films are comprised of a plurality of channels with V-shaped or rectangular cross-sections, and combinations of these, as well as structures that have secondary channels, i. e., channels within channels. For open channels, the desired surface energy of the microstructured surface of V-channeled fluid control films is such that:

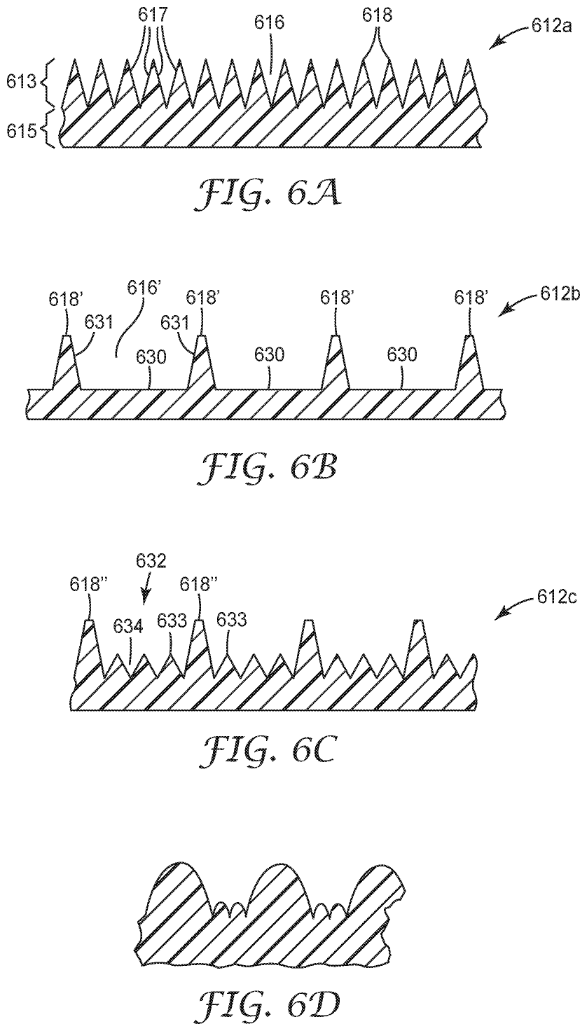

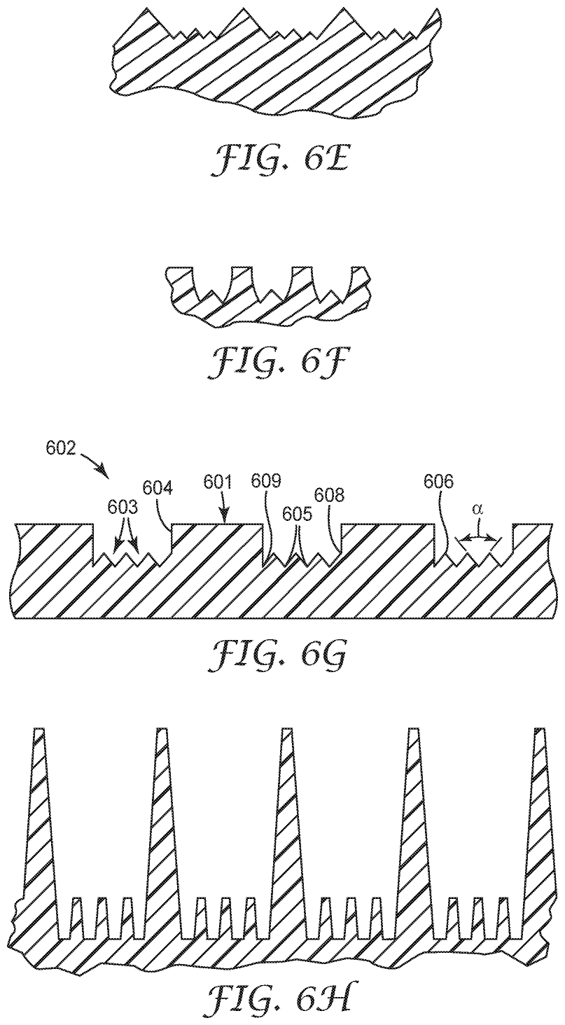

Theta<(90−Alpha/2),

wherein Theta is the contact angle of the liquid with the film and Alpha (α) is the average included angle of the secondary V-channel notches. (See, e. g., FIG. 6G).

Any suitable known method may be utilized to achieve a hydrophilic surface on fluid control films. Surface treatments may be employed such as topical application of a surfactant, plasma treatment, vacuum deposition, polymerization of hydrophilic monomers, grafting hydrophilic moieties onto the film surface, corona or flame treatment, etc. Alternatively, a surfactant or other suitable agent may be blended with the resin as an internal additive at the time of film extrusion. It is typically preferred to incorporate a surfactant in the polymeric composition from which the fluid control film is made rather than rely upon topical application of a surfactant coating. Topically applied coatings tend to fill in, i. e., blunt, the notches of the channels, thereby interfering with the desired liquid flow to which the invention is directed. An illustrative example of a surfactant that can be incorporated in polyethylene fluid control films is TRITON™ X-100, an octylphenoxypolyethoxyethanol nonionic surfactant, e. g., used at between about 0.1 and 0.5 weight percent. An illustrative method for surface modification of the films is the topical application of a 1 percent aqueous solution of the reaction product comprising 90 weight percent or more of:

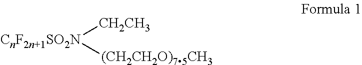

wherein n=8 (97 percent), n=7 (3 percent), and 10 weight percent or less of CH2CH3 CnF2n+1 S02NsH

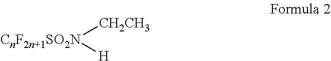

wherein n=8 (97 percent), n=7 (3 percent). Preparation of such agents is disclosed in U.S. Pat. No. 2,915,554 (Ahlbrecht et al.).

As discussed above, a surfactant or mixture of surfactants may be applied to the surface of the fluid control film or impregnated into the article in order to adjust the properties of the fluid control film or article. For example, it may be desired to make the surface of the fluid control film more hydrophilic than the film would be without such a component.

Desirably the favorable fluid transport properties are retained throughout the life of the product into which the fluid control film is incorporated. In order to ensure the surfactant is available throughout the life of the fluid control film the surfactant preferably is available in sufficient quantity in the article throughout the life of the article or is immobilized at the surface of the fluid control film. For example, a hydroxyl functional surfactant can be immobilized to a fluid control film by functionalizing the surfactant with a di- or tri-alkoxy silane functional group. The surfactant could then be applied to the surface of the fluid control film or impregnated into the article with the article subsequently exposed to moisture. The moisture would result in hydrolysis and subsequent condensation to a polysiloxane. Hydroxy functional surfactants (especially 1,2 diol surfactants) may also be immobilized by association with borate ion. Suitable surfactants include anionic, cationic, and non-ionic surfactants, however, nonionic surfactants may be preferred due to their relatively low irritation potential. Polyethoxylated and polyglucoside surfactants are particularly preferred including polyethoxylated alkyl, aralkyl, and alkenyl alcohols, ethylene oxide and propylene oxide copolymers such as “Pluronic” and “Tetronic”, alkylpolyglucosides, polyglyceryl esters, and the like. Other suitable surfactants are disclosed in Ser. No. 08/576,255.

As discussed above, a hydrophilic polymer or mixture of polymers may be applied to the surface of the fluid control film or impregnated into the article in order to adjust the properties of the fluid control film. In order to ensure the hydrophilic polymer is available throughout the life of the fluid control film the polymer preferably is available in sufficient quantity in the article throughout the life of the article or is immobilized at the surface of the fluid control film. Alternatively, a hydrophilic monomer may be added to the article and polymerized in situ to form an interpenetrating polymer network. For example, a hydrophilic acrylate and initiator could be added and polymerized by heat or actinic radiation.

Suitable hydrophilic polymers include: homo and copolymers of ethylene oxide; hydrophilic polymers incorporating vinyl unsaturated monomers such as vinylpyrrolidone, carboxylic acid, sulfonic acid, or phosphonic acid functional acrylates such as acrylic acid, hydroxy functional acrylates such as hydroxyethylacrylate, vinyl acetate and its hydrolyzed derivatives (e. g. polyvinylalcohol), acrylamides, polyethoxylated acrylates, and the like; hydrophilic modified celluloses, as well as polysaccharides such as starch and modified starches, dextran, and the like.

As discussed above, a hydrophilic silane or mixture of silanes may be applied to the surface of the fluid control film or impregnated into the film in order to adjust the properties of the fluid control film. Suitable silanes include the anionic silanes disclosed in U.S. Pat. No. 5,585,186, as well as non-ionic or cationic hydrophilic silanes. Cationic silanes may be advantageous in that certain of these silanes are also believed to have antimicrobial properties.

As previously mentioned the channels of fluid control films can be of any geometry that provides desired liquid transport. Fluid control films can have primary channels on both major surfaces, however it has been found that fluid control films having primary channels on only one major surface (e. g. as shown in FIGS. 6A-6C and 6G) are more favorable for use of the absorbent articles and liquid conducting members thereof.

As shown in FIG. 6A, channels 616 can be defined within the layer 612a in accordance with the illustrated embodiment by a series of v-shaped sidewalls 617 and peaks 618. In some cases, the sidewalls 617 and peaks 618 may extend entirely from one edge of the layer 612 to another without alteration although, in some applications, it may be desirable to shorten the sidewalls 617 and thus extend the peaks 618 only along a portion of the structured surface 613. That is, channels 616 that are defined between peaks 618 may extend entirely from one edge to another edge of the layer 612, or such channels 616 may only be defined to extend over a portion of the layer 612. Channels that extend only over a portion may begin at an edge of the layer 612, or they may begin and end intermediately within the structured surface 613 of the layer 612. The channels are defined in a predetermined, preferably ordered arrangement over a continuous surface of polymeric material. Layer 612 further comprises a backing 615.

As shown in FIG. 6B, channels 616′ of layer 612b have a wider flat valley between slightly flattened peaks 618′. Like the FIG. 6A embodiment, a cap layer can be secured along one or more of the peaks 618′ to define discrete channels 616′. In this case, bottom surfaces 630 extend between channel sidewalls 631, whereas in the FIG. 6A embodiment, sidewalls 617 connect together along lines.

FIG. 6C illustrates a configuration where wide channels 632 of layer 612c are defined between peaks 618″, but instead of providing a flat surface between channel sidewalls, a plurality of smaller peaks 633 are located between the sidewalls of the peaks 618″. These smaller peaks 633 thus define secondary channels 634 therebetween. Peaks 633 may or may not rise to the same level as peaks 618, and as illustrated create a first wide channel 632 including smaller channels 634 distributed therein. The peaks 618″ and 633 need not be evenly distributed with respect to themselves or each other.

FIGS. 6D-6H illustrate various alternative embodiments of the fluid control film. Although FIGS. 6A-6H illustrate elongated, linearly-configured channels, the channels may be provided in other configurations. For example, the channels could have varying cross-sectional widths along the channel length—that is, the channels could diverge and/or converge along the length of the channel. The channel sidewalls could also be contoured rather than being straight in the direction of extension of the channel, or in the channel height. Generally, any channel configuration that can provide at least multiple discrete channel portions that extend from a first point to a second point within the fluid transport device are contemplated. The channels may be configured to remain discrete along their whole length if desired.

With reference to FIG. 6G, one preferred geometry is a rectilinear primary channel 602 in a flat film 601. The primary channel 602 has sidewalls 604 and included secondary channels 603 which form a multitude of notches 605. The notches 605 (or secondary channels 603, where the channels are V-shaped and have substantially straight sidewalls) have an included angle of (i. e., angle Alpha) from about 10 degrees to about 120 degrees, preferably from about 10 degrees to about 100 degrees, and most preferably from about 20 to about 95 degrees. The notch included angle is generally the secant angle taken from the notch to a point 2 to 1000 microns from the notch on the sidewalls forming the notch, preferably the included angle is the secant angle taken at a point halfway up the secondary channel sidewalls. It has been observed that notches with narrower included angular widths generally provide greater vertical wicking distance. However, if Alpha is too narrow, the flow rate will become significantly lower. If Alpha is too wide, the notch or secondary channel may fail to provide desired wicking action. As Alpha gets narrower, the contact angle of the liquid need not be as low, to get similar liquid transport, as the contact angle must be for notches or channels with higher angular widths.

The primary channel included angle is not critical except in that it should not be so wide that the primary channel is ineffective in channeling liquid. Generally, the primary channel maximum width is less than 3000 microns and preferably less than 1500 microns. The included angle of a V-channel shaped primary channel will generally be from about 10 degrees to 120 degrees, preferably 30 to 90 degrees. If the included angle of the primary channel is too narrow, the primary channel may not have sufficient width at its base so that it is capable of accommodating an adequate number of secondary channels. Generally, it is preferred that the included angle of the primary channel be greater than the included angle of the secondary channels so as to accommodate the two or more secondary channels at the base of the primary channel. Generally, the secondary channels have an included angle at least 20 percent smaller than the included angle of the primary channel (for V-shaped primary channels).

With reference to FIG. 6g, the depth of the primary channels (602, 622) (the height of the peaks or tops above the lowermost channel notch), “d”, is substantially uniform, and is suitably from about 5 to about 3000 microns, typically from about 50 to about 3000 microns, preferably from about 75 to about 1500 microns, and most preferably is from about 100 to about 1000 microns. It will be understood that in some embodiments films with channels (602, 622) having depths larger than the indicated ranges may be used. If the channels are unduly deep, the overall thickness of the fluid control film will be unnecessarily high and the film may tend to be stiffer than is desired. The width of the primary channel at its base may be sufficient to accommodate two or more secondary channels.

As illustrated in FIGS. 6g, in each primary channel (602, 622) are at least two secondary channels (603, 623) and at least two notches (605, 625), the notch (605, 625) or notches of each secondary channel (603, 623) is separated by a secondary peak (606, 626). Generally, each secondary channel will generally have only one notch, but a secondary channel will have two notches if the secondary channel is rectangular. The secondary peak (606, 626) for V-channel shaped secondary channels is generally characterized by an included angle Beta (β) which is generally equal to (α¹+α²)/2 where α¹ and α² are the included (Alpha) angles of the two adjacent V-channel shaped secondary channels (603, 623), assuming that the two sidewalls forming each secondary channel are symmetrical and not curved. Generally, the angle β would be from about 10 to about 120 degrees, preferably from about 10 to about 90 degrees, and most preferably from about 20 to about 60 degrees. The secondary peak could also be flat (in which case the included angle would theoretically be 0 degrees) or even curved, e. g., convex or concave, with no distinct top or included angle. Preferably, there are at least three secondary channels (603, 623) and/or at least three notches for each primary channel (602, 622), included any notches (605, 625) associated with the end channels (notches 608 or 609) as shown in FIG. 6G.

The depth of one of the secondary channels (603, 623) (the height of the top of the secondary peaks 606 over the notches 605) is uniform over the length of the fluid control films, and is typically at least 5 microns. The depth of the secondary channels (603, 623) is generally 0.5 to 80 percent of the depth of the primary channels, preferably 5 to 50 percent. The spacing of the notches (605, 625) on either side of a peak 6 is also preferably uniform over the length of the fluid control film. Preferably the primary and/or secondary channel depth and width varies by less than 20 percent, preferably less than 10 percent for each channel over a given length of the fluid control film. Variation in the secondary channel depth and shape above this range has a substantial adverse impact on the rate and uniformity of liquid transport along the fluid control film. Generally, the primary and secondary channels are continuous and undisturbed.

The structured surface can also be provided with a very low profile. Thus, elongate liquid conducting members comprising are contemplated where the structured polymeric layer of the fluid control film has a thickness of less than 5000 micrometers, and possibly less than 1500 micrometers. To do this, the channels may be defined by peaks that have a height of approximately 5 to 1200 micrometers and that have a peak distance of about 10 to 2000 micrometers.

Suitable channels in fluid control films used in the liquid conducting members of the present invention may be of any suitable geometry but are generally rectangular (typically having depths of 50 to 3000 micron and widths of 50 to 3000 micron or “V” channel patterns (typically having depths of about 50 to 3000 micron and heights of 50 to 3000 micron) with an included angle of generally 20 to 120 degrees and preferably about 45 degrees. The presently preferred structure has a nested construction wherein the master channels are 200 micron deep and repeat every 225 micron with three equally spaced channels in the base each 40 micron deep. Compound channels are also possible and often preferably such as rectangular channels that contain smaller rectangular or V channels within.

Elongate liquid conducting members may comprise layers of two or more fluid control films. As indicated above, elongate liquid conducting members comprising a fluid control film may favorably comprise a cap layer to enclose the channels, at least in part along the length of the liquid conducting member, for example to enhance creation of discrete channel and more particularly to minimize or prevent fluid from being transferred over the sides of the liquid conducting members along its length for example onto the topsheet in the peripheral region. A cap layer may be juxtaposed against the structured surface, in particular onto the highest peaks or plateaus of the structure. A cap layer may be bonded to some or all the highest peaks or plateaus of the structured surface. This can be done thermally or by using conventional adhesives that are compatible with the cap layer material and the polymeric structured layer. Bonds may be provided entirely along the peaks or plateaus to the cap layer or the bonds may be spot welds or bonds that may be placed thereon in an ordered or random pattern. Cap layer preferably is made from a polymeric material such as the polymers described for the structured polymeric layer. Polymers may be chosen such that the cap layer can be secured to the structured surface without using an adhesive. Such a polymer could be chosen such that the cap layer becomes securely welded to the structured surface by applying heat, for example, as from an ultrasonic welding operation. Optionally, dependent on the overall absorbent article construction, a cap layer may comprise a material such as a spunlaced, spunbond, blown microfiber or carded nonwoven.

Sidewalls may be arranged surrounding the liquid conducting members in part or entirely. These sidewalls may form a U-shaped profile. Such profiles may be closed on one end of the liquid conducting members.

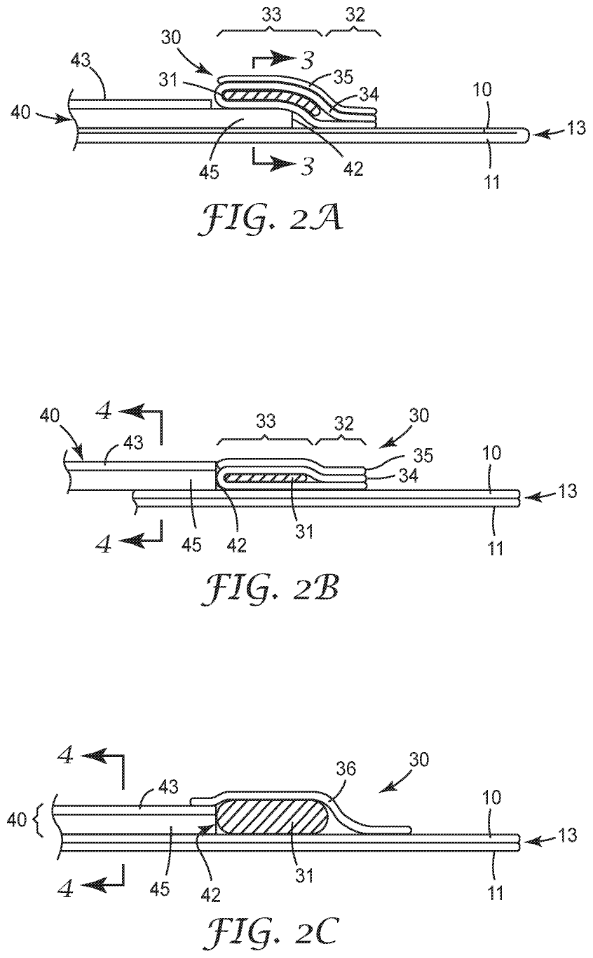

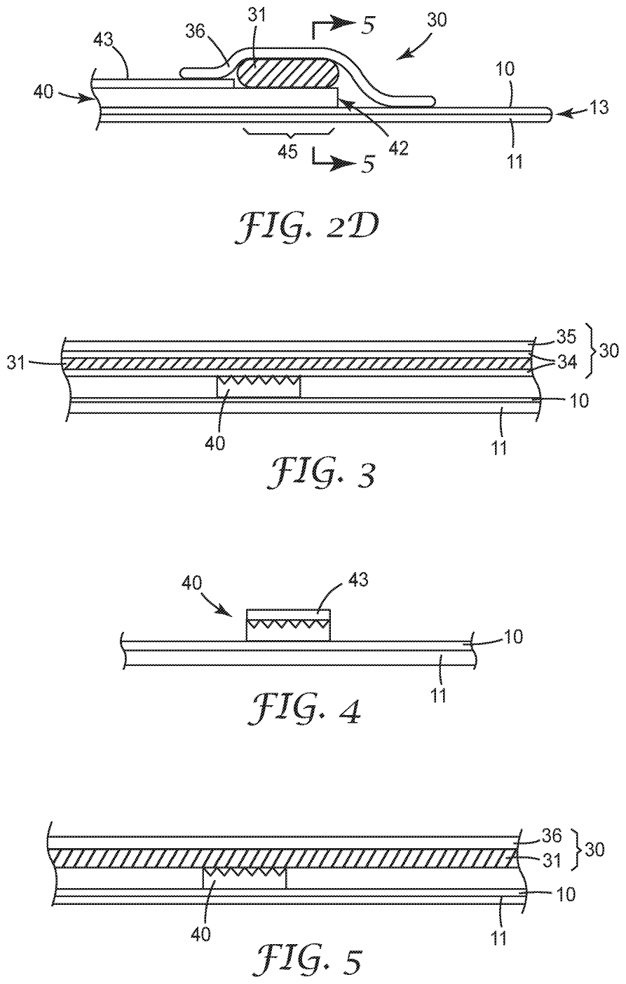

Returning to the exemplary embodiment of FIG. 1, it can be recognized that each of the liquid conducting members 40, which comprise a fluid conduct film, have a cap layer 43 wherein the end portions 44 of the members near the first end 41 remain uncapped, in order to facilitate wicking liquid from the central region 21 of the absorbent article 100 into and along the channels of the fluid conducting film. FIGS. 2A to D provide enlarged cross-sectional views of four exemplary variants of the exemplary absorbent article of FIG. 1, in particular four variants of the liquid response barrier member 30 and the end portion 45 of a liquid conducting member 40 near its second end 42.

In reference to FIG. 2A, it can be recognized that the cap layer 43 does not extend to the second end 42 of the liquid conducting member, i. e. the second end portion 45 of the member near its second end is uncapped. The second end portion 45 of liquid conducting member 40 is disposed under the liquid response barrier member 30 and thus between the topsheet 10 and the liquid response barrier member 30.

The elongate liquid response barrier member 30 has a longitudinal attachment edge portion 32 near the outer edge, here the rear waist edge 13, of the absorbent article. The liquid response barrier member is attached along the longitudinal attachment edge portion to the interior surface of the absorbent article, in particular to the topsheet. (As typical in the art the topsheet 10 is bonded to the backsheet 11 is the peripheral region of the absorbent article). Absorbent material 31 is disposed in the free portion 33 (i. e. unattached portion) of the liquid response barrier member 30. The free portion 33 of the elongate liquid response barrier member 30 can lie towards the topsheet, and in this exemplary embodiment lie resting on the second end portion 45 of liquid conducting member 40. The free portion 33 of the elongate liquid response barrier member 30 can also pivot about an axis defined by the attachment edge portion 32. As shown in this exemplary embodiment, the absorbent material may be provided in a sheath 34 made of a liquid permeable material. The sheath may comprise a woven or a nonwoven web adapted to readily take in and transport liquid (for example having hydrophilic properties) as well as being extensible. The liquid response barrier member may optionally be provided with an outer liquid impermeable layer 35, distant to the liquid conducting member and near to the wearer of the article in use. Any liquid impermeable material may be used, favorably a soft material and a material is also adapted to expand. The portion of the liquid permeable sheath 34 resting on the liquid conducting member 40 is in fluid communication with the conducting member and thus liquid can be communicated from the liquid conducting member 40 to the absorbent material 31 over that portion of liquid permeable sheath.

Referring to FIG. 3, it can be recognized that the portion of the liquid permeable sheath 34 of the liquid response barrier member 30 which is in contact with the liquid conducting member 40, rests on top of the highest peaks (or if applicable plateaus) of the fluid control film, thereby capping the channels in the second end portion of the liquid conducting member and enhancing wicking and capillary flow. Although fluid control film shown in this Figure and other Figures of other exemplary embodiments and variants has a structured surface comprising multiple V-shaped peaks (e. g., as shown in FIG. 6A), other configurations are contemplated.

FIG. 2B shows a cross-sectional cutaway view of an exemplary variant having the same type of liquid response barrier member 30 as the exemplary variant shown in FIG. 2A, but with an alternative configuration with respect to the positioning and construction of the second end portion 45 of liquid conducting member 40. In this embodiment, the longitudinal attachment edge portion 32 is here once again attached near the outer edge, i. e. the rear waist edge 13, of the absorbent article and now the free portion 33 of the elongate liquid response barrier member 30 rests on the topsheet 10. Also in this embodiment the cap layer 43 extends to the second end 42 of the liquid conducting member 40. From FIG. 4, it can be recognized that the cap layer 43, rests on top of the highest peaks (or if applicable plateaus) of the fluid control film. The second end 42 of the liquid conducting member 40 is positioned to abut the interior end of the free portion of the liquid response barrier member. Again, the portion of the liquid permeable sheath 34 in contact with the liquid conducting member 40 is in fluid communication with the conducting member and its channel and thus liquid can be communicated from the liquid conducting member 40 to the absorbent material 31 over that portion of liquid permeable sheath. In such configurations it may be favorably to use a liquid conducting member comprising a stack of fluid control films.

For both exemplary variants shown in FIGS. 2A and 2B, once the absorbent material 31 expands due to absorption of liquid to the extent that the free portion 33 pivots up and outwardly away from the topsheet 10, the liquid response barrier 30 will form a barrier between the elongate liquid response barrier member and a portion of the body of a wearer of the absorbent article. Advantageously once the free portion 33 pivots to an extent that the free portion is no longer in fluid communication with the second end 42 or second end portion 45 of the liquid conducting member 40, active drawing or pulling of liquid from liquid conducting member by the liquid barrier response member 30 will cease, thereby favorably minimizing any undesired liquid flow once the free portion is no longer in fluid communication with the liquid conducting member.

FIG. 2C shows a cross-sectional cutaway view of an exemplary variant having the different type of liquid response barrier member 30, but with a similar configuration with respect to the positioning and construction of the second end portion 45 of liquid conducting member 40 as the exemplary variant shown in FIG. 2B. In this embodiment, the liquid response barrier member 30 comprises absorbent material 31 and a cover layer 36. Favorably the cover layer 36 comprises a liquid impermeable material and is extensible. Also in this embodiment the cap layer 43 extends to the second end 42 of the liquid conducting member 40. The absorbent material 31 of the liquid response barrier member is disposed between the cover layer 36 and the top sheet 10 and the edges or edge portions of the cover layer are attached to the topsheet and cap layer 43 in the second end portion 45 of liquid conducting member.

The second end 42 of the liquid conducting member is positioned 40 to abut the absorbent material 31 and thus the absorbent material is in direct fluid communication with the channels of the fluid control film of the liquid conducting member 30 (see e. g. FIG. 4). Also in such configurations it may be favorably to use a liquid conducting member comprising a stack of fluid control films.

FIG. 2D shows a cross-sectional cutaway view of an exemplary variant having the same type of liquid response barrier member 30 as in the exemplary embodiment of FIG. 2C, but a different configuration with respect to the positioning and construction of the second end portion 45 of liquid conducting member 40. Similar to the exemplary embodiment of FIG. 2C, the liquid response barrier member 30 comprises absorbent material 31 and a cover layer 36, favorably a liquid impermeable cover layer, and the edges or edge portions of the cover layer are attached to the topsheet 10 and the cap layer 43 of liquid conducting member. In this exemplary embodiment, the second end portion 45 of liquid conducting member 40 is uncapped and disposed under the absorbent material 31. In other words, the absorbent material 31 is disposed between the cover layer 36 and the second end portion 45 of the liquid conducting member 40. Referring to FIG. 5, the absorbent material 31 of the liquid response barrier member 30 extends over the structures of the fluid control film of the liquid conducting member 40.

For both exemplary variants shown in FIGS. 2C and 2D, once the absorbent material 31 expands due to absorption of liquid, the liquid response barrier 30 will form a barrier or a seal between the elongate liquid response barrier member and a portion of the body of a wearer of the absorbent article. Advantageously once absorbent material completely expands, i. e. the absorbent material is saturated, active drawing or pulling of liquid from liquid conducting member 30 by the liquid barrier response member 30 will either cease or be significantly reduced. These in turn aid in minimizing any undesired liquid flow from the second end portion 45 of the liquid conducting member 40 e. g. on to the topsheet 10 in the vicinity of the liquid response barrier member 30. This effect can be enhanced by optimizing the amount of absorbent material in the liquid response barrier member having regard to the absorption capacity of the particular absorbent material being used, so that the amount is used sufficient to achieve desired extent of expansion to achieve the desired barrier and/or seal effect while avoiding or significantly minimizing the use of excess amounts of absorbent material. Also, the use of a liquid impermeable cover layer 26 is particularly advantageous in that fluid communication from the interior of the liquid response barrier member 30 to the exterior will be avoided or at least significantly minimized. In addition to a liquid impermeable cover layer 36 will aid in containing any residual liquid that enters into the interior of the liquid response barrier member 30 from the second end portion 45 of the liquid conducting member 40, especially since the edge or edge portions of the cover layer 36 is attached to the topsheet 10 and the topsheet is bonded to the backsheet 11, which is normally also liquid impermeable.

Returning to FIG. 8 which shows an illustrative exemplary embodiment, in which the liquid conducting members 40 are disposed between the topsheet 10 and the backsheet 11. The outer surface of the liquid conducting member 40 that is near the backsheet 11 is favorably attached or bonded to the interior surface of the backsheet at least in the peripheral region 21 of the absorbent article 100. In the central region the liquid conducting members may also be attached or bonded to the backsheet and thus be disposed between absorbent core 20 and the backsheet 11 or alternatively the portion of the elongate liquid conducting member 40 in the central region may be disposed in the absorbent core 20 or between the topsheet 10 and the absorbent core 20.

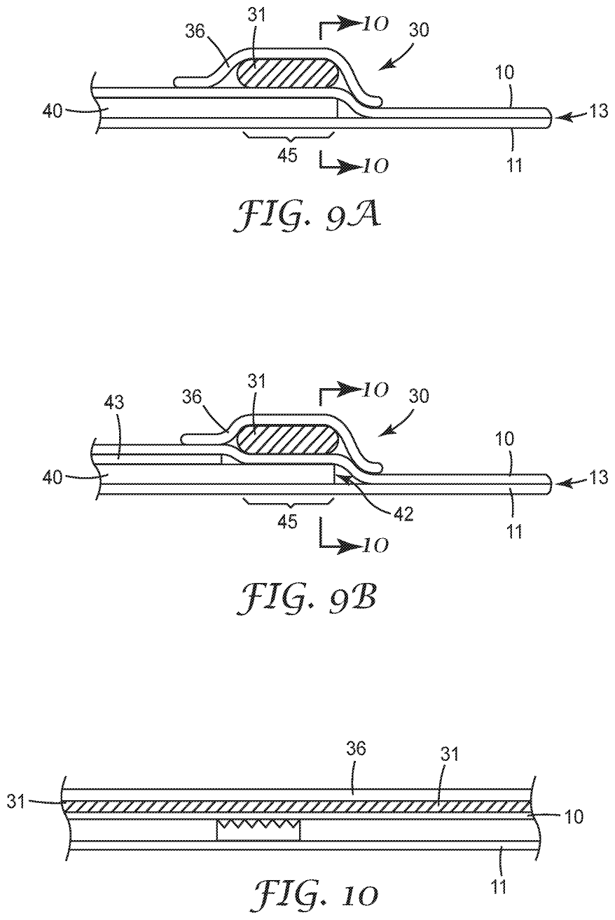

FIGS. 9A and B provide enlarged cross-sectional views of two exemplary variants of the exemplary absorbent article of FIG. 8. Similar to the exemplary embodiments of FIGS. 2C and D, the liquid response barrier member 30 comprises absorbent material 31 and a cover layer 36, favorably a liquid impermeable cover layer. The edges or edge portions of the cover layer are attached to the topsheet 10. The liquid conducting member 40 is positioned under the topsheet 10, such that the second end portion 45 is located underneath the liquid response barrier member 30 and in particular its absorbent material 31. At least that portion of the top sheet 10 that is adjacent or proximate to the second end portion 45 of the liquid conducting member 40 is adapted so that the topsheet 10 is in fluid communication with the liquid conducting member 40 and thus liquid can be communicated from the liquid conducting member 40 to the absorbent material 31. For example when using a conventional topsheet, e. g. a topsheet made of a hydrophobic material or is treated to be hydrophobic in order to isolate the wearer's skin and when at least a portion of the upper surface is to treated to be hydrophilic to liquids will transfer through the topsheet to the absorbent core of the absorbent article, at least that the portion of the top sheet 10 that is adjacent or proximate to the second end portion 45 of the liquid conducting member 40 is favorably provide with one or more apertures. Referring to FIG. 10, it can be recognized that the topsheet 10 is disposed on top of the highest peaks (or if applicable plateaus) of the fluid control film of the liquid conducting member and that the absorbent material 31 of the liquid response barrier member 30 is disposed on the topsheet 10. In the exemplary embodiment variant of FIG. 9A, the liquid conducting member 40 does not include a cap layer. In an embodiment variant of the FIG. 9B, the liquid conducting member 40 includes a cap layer 43, wherein the second end portion 45 of the liquid conducting member 40 is uncapped and similar to the previously described exemplary embodiments the first end portion of liquid conducting member 40, in particular the complete portion of the liquid conducting member that is located in the central region of the absorbent article, is also uncapped e. g. to facilitate wicking.

Absorbent material of the liquid response barrier member may comprise super absorbents such as those described in U.S. Pat. No. 7,314,967. Absorbent material of the liquid response barrier member may comprise absorbent gelling material (such as Gelling Elastic Material produced by The Procter and Gamble Corp; Gelling adhesive material manufactured by H. B. Fuller of St. Paul, Minn., USA, under the designation HydroLock, or superporous hydrogels manufactured by Akina West of Lafayette, Ind., USA, under the designation Aquagel) or a fluid stable aggregates, or any combinations thereof.

The absorbent gelling material (“AGM”) includes a variety of water-insoluble, but water-swellable polymers capable of absorbing large quantities of fluids. Such polymer materials are generally known in the art and include all those well-known polymers used or deemed useful in the context of disposable absorbent article technology. Particularly the AGMs disclosed in EP-A-752 892 or those disclosed in the textbook entitled “Modem Super Absorbent Technology” by F. L. Buchholz and A. T. Graham, published by Wiley VCH, New York, 1998 are useful. AGM particles may be of numerous shapes. The term “particles” refers to granules, fibers, flakes, spheres, powders, platelets, and other shapes and forms known to person skilled in the art of AGMs. The particles can be in the form of granules, beads, that have a particle size from about 10 μ]m to about 1000 μm, or even from about 100 μm to about 1000 μm, or even from about 150 μm to about 850 μm and or even from about 150 μm to about 500 μm. In another embodiment, the AGMs can be in the shape of fibers, i. e. elongated, acicular AGM particles. In another embodiment, the AGM may be pre-wetted such that the AGM is already in a gel like state. The fibers can also be in the form of a long filament that can be woven into a sheet. The AGM may be in sheet form and disposed on the second end portion of the liquid conducting member or the topsheet. Alternatively, the AGM may be printed or glued to the cover layer, in particular to the liquid impermeable cover layer using any suitable bonding or printing process that is well known in the art.

Fluid stable aggregates (“FSA's”) can be used to make up absorbent polymeric macrostructures. Exemplary FSA's structures suitable for use are described in U.S. Pat. No. 5,536,264 entitled “Absorbent Composites Comprising a Porous Macrostructure of Absorbent Gelling Particles and A Substrate” issued to Hsueh et al. on Jul. 16, 1996; U.S. Pat. No. 6,224,961 entitled “Absorbent Macrostructure Made From Mixtures of Different Hydrogel-Forming Absorbent Polymers for Improved Fluid Handling Capability” issued to Hsueh et al. on May 1, 2001; U.S. Pat. No. 5,428,076 entitled “Flexible, Porous, Absorbent, Polymeric Macrostructures and Methods of Making the Same” issued to Roe on Jun. 27, 1995; U.S. Pat. No. 5,372,766 entitled “Flexible, Porous, Absorbent, Polymeric Macrostructures and Methods of Making the Same” issued to Roe on Dec. 13, 1994; U.S. Pat. No. 5,324,561 entitled “Porous, Absorbent, Macrostructures of Bonded Particles Surface Crosslinked with Cationic Amino-Epichlorohydrin Adducts” issued to Rezai et al. on Jun. 28, 1994; U.S. Pat. No. 5,124,188 entitled “Porous, Absorbent, Polymeric Macrostructures and Methods of Making the Same” issued to Roe et al. on Jun. 23, 1992; and U.S. Pat. No. 5,102,597 entitled “Porous, Absorbent, Polymeric Macrostructures and Methods of Making the Same” issued to Roe et al. on Apr. 7, 1992.

Any suitable liquid impermeable material known in the art may be used in a liquid impermeable layer or cover layer of the liquid response barrier member. For example, a liquid impermeable layer or cover layer may comprise an elastomeric material which includes those materials manufactured by Kraton Polymers Inc. of Houston, Tex., USA, and sold under trade names Kraton D and Kraton G.

Any suitable liquid permeable material known in the art may be used in a liquid permeable layer or sheath of the liquid response barrier member. As an example, the liquid permeable layer or sheath may comprise a carded nonwoven material that is made of bi-component fibers of a polyethylene (PE) and a polypropylene (PP) where the ratio of PE/PP is about 50/50 (e. g. obtainable from Chisso Corp., Moriyama, Japan). Alternatively, the liquid permeable sheath may comprise a spunbonded nonwoven material that is made of bi-component fibers of a polyethylene (PE) and a polypropylene (PP) with a ratio of PE/PP of about 80/20 (e. g. obtainable from Mitsui Petrochemical Industries, Ltd., Tokyo, Japan).

Exemplary embodiments of this invention are discussed and reference has been made to some possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the claims provided below and equivalents thereof.

### LIST REFERENCES NUMBERS in FIGS. 1 to 5 and 8 to 10

100 absorbent article
10 topsheet
11 backsheet
12 peripheral region
13 rear waist edge
14 front waist edge
15 longitudinal edges
20 absorbent core
21 central region
30 liquid response barrier member
31 absorbent material of liquid response barrier member
32 attachment edge
33 free portion
34 sheath
35 liquid impermeable layer
36 cover layer
40 liquid conducting member
41 first end
42 second end
43 cap layer
44 first end portion
45 second end portion

1. An absorbent article having a central region and a peripheral region, the absorbent article comprising:
a liquid permeable topsheet that is located towards the wearer with the absorbent article is in use;
a backsheet;
an absorbent core disposed in the central region between the top sheet and the backsheet;
at least one elongate liquid response barrier member disposed on an interior surface of the absorbent article in the peripheral region and positioned along an outer edge of the absorbent article, either adjacent to or spaced apart from said outer edge, the at least one elongate liquid response barrier member comprising absorbent material and being configured and arranged such that the absorbent material expands upon absorbing liquid and upon expansion of the absorbent material the at least one elongate liquid response barrier member forms a barrier or a seal between the at least one elongate liquid response barrier member and a portion of the body of a wearer of the absorbent article; and
wherein the absorbent article further comprises at least one elongate liquid conducting member extending from the central region of the absorbent article to the at least one elongate liquid response barrier member.

2. An absorbent article according to claim 1, wherein a first end of the at least one elongate liquid conducting member positioned in the central region or directly adjacent to the outer periphery of the central region and either the second end or a portion including the second end of the at least one elongate liquid conducting member is positioned adjacent or in proximity to the at least one elongate liquid response barrier member such that fluid that is conducted along the at least one elongate liquid conducting member is communicated to the absorbent material of the at least one elongate liquid response barrier member.

3. An absorbent article according to claim 1, wherein the at least one elongate liquid conducting member is disposed either between the topsheet and the backsheet or on the interior surface of the absorbent article.

4. An absorbent article according to claim 1, wherein the at least one elongate liquid conducting members comprises a fluid control film having a microstructure-bearing surface with a plurality of channels therein that permit transport of liquid between a central portion and the at least one elongate liquid response barrier member.

5. An absorbent article according to claim 4, wherein the microstructure-bearing surface is hydrophilic.

6. An absorbent article according to claim 4, wherein the channels have a cross-sectional geometry comprising V-shaped channels, rectangular-shaped channels, or a combination of V- and rectangular-shaped channels.

7. An absorbent article according to claim 4, wherein the channels are between about 5 and about 3000 microns deep.

8. An absorbent article according to claim 4, wherein the fluid control film comprises a plurality of primary channels having at least two secondary channels, each of the secondary channels forming at least one notch, wherein the primary channels have a depth of from 50 to 3000 microns and the depth of the secondary channels is from 5 to 50 percent of the depth of the primary channels.

9. An absorbent article according to claim 4, wherein the channels have an included angle between about 10 degrees and about 120 degrees.

10. An absorbent article according to claim 4, wherein the liquid conducting member comprises a cap layer to enclose the channels, at least in part along the length of the liquid conducting member.

11. An absorbent article according to claim 10, wherein a first end portion or the complete portion of the at least one elongate liquid conducting member located in the central region of the absorbent article is free of the cap layer.

12. An absorbent article according to claim 10, wherein either (i) the complete portion of the at least one elongate liquid conducting member located in the peripheral central region includes the cap layer or (i) the portion of the at least one elongate liquid conducting member that is located in the peripheral central region with the exception of the end portion that is located adjacent or in proximity to the at least one elongate liquid response barrier member includes a cap layer.

13. An absorbent article according to claim 4, wherein the channels comprise a thermoplastic material selected from the group consisting of polyolefins, polyesters, polyamides, poly(vinyl chloride), polyether esters, polyimides, polyesteramides, polyurethanes, polyacrylates, polyvinylacetate, hydrolyzed derivatives of polyvinyl acetate and combinations thereof.

14. An absorbent article according to claim 4, wherein the channels comprise a thermoset material selected from the group consisting of polyurethanes, acrylates, epoxies and silicones.

15. An absorbent article according to claim 1, wherein the at least one elongate liquid response barrier member has an longitudinal attachment edge portion near said outer edge of the absorbent article and the at least one elongate liquid response barrier member is attached along the longitudinal attachment edge portion to an interior surface of the absorbent article, so that the free portion of the at least one elongate liquid response barrier member can lie towards the topsheet and also pivot about an axis defined by the attachment edge portion and wherein the absorbent material is located in the free portion of the at least one elongate liquid response barrier member.

16. An absorbent article according to claim 15, wherein prior to expansion of the absorbent material, either the free end of the free portion of the at least one elongate liquid response barrier member abuts an end of the at least one elongate liquid conducting member or the free portion of the at least one elongate liquid response barrier member lies on an end portion of the at least one elongate liquid conducting member.

17. An absorbent article according to claim 15, where the absorbent material is disposed within a liquid permeable sheath or on a liquid permeable layer.

18. An absorbent article according to claim 15, wherein at least the free portion of the at least one elongate liquid response barrier member comprises a liquid impermeable layer and a liquid permeable layer, wherein the liquid impermeable layer is distant to the topsheet and the liquid permeable layer is near the topsheet and the absorbent material is disposed between the liquid impermeable layer and liquid permeable cover layer.

19. An absorbent article according to claim 1, wherein the at least one elongate liquid response barrier member further comprises a cover layer and the outer edges or edge portions of the cover layer are attached to the interior surface of the absorbent article, wherein the absorbent material either (i) is disposed between the cover layer and an end portion of the at least one elongate liquid conducting member or (ii) is disposed between the cover layer and the topsheet and abuts an end of the at least one elongate liquid conducting member

20. An absorbent article according to claim 19, wherein the cover layer is liquid impermeable.

21. An absorbent article according to claim 19, wherein an enclosed space is provided between the cover layer and the interior surface housing the absorbent material and either said end or end portion of the at least one elongate liquid conducting member.
